(19) United States
(12) Patent Application Publication
(10) Pub. No.: US 2021/0374631 A1
(43) Pub. Date: Dec. 2, 2021

(54) PROCESS FOR WORKPLACE DISEASE MANAGEMENT

(71) Applicant: The Modern Group, Ltd., Beaumont, TX (US)

(72) Inventor: Robert LUKE, Youngesville, LA (US)

(21) Appl. No.: 17/326,163

(22) Filed: May 20, 2021

Related U.S. Application Data
(60) Provisional application No. 63/029,766, filed on May 26, 2020.

(51) Int. Cl. G06Q 10/06; G06Q 10/10; G16H 50/30; G16H 50/20; G16H 10/20; G16H 10/40; G16H 50/80; G16H 10/65; G07C 9/38; G16H 40/20

(52) U.S. Cl. CPC G06Q 10/0635; G06Q 10/105; G16H 50/30; G16H 50/20; G16H 10/20; G06K 9/628; G16H 50/80; G16H 10/65; G06Q 10/1091; G07C 9/38; G16H 40/20; G16H 10/40

(57) ABSTRACT

A method for workplace disease management may include determining employer criteria, screening employees, classifying employees into risk groups, identifying symptomatic employees, establishing work paths and work areas, and monitoring employee compliance with work paths and work areas.

### CROSS-REFERENCE TO RELATED APPLICATIONS

This application is a nonprovisional application which claims priority from U.S. provisional application No. 63/029,766, filed May 26, 2020, which is incorporated by reference herein in its entirety.

### TECHNICAL FIELD/FIELD OF THE DISCLOSURE

The present disclosure relates to methods for workplace disease management.

### BACKGROUND OF THE DISCLOSURE

Managing disease outbreaks, such as virus pandemics and other persistent communicable infectious agents, has traditionally been addressed by encouraging infected employees to stay home, limited operations by the employer, social distancing of employees, and face or body covering to limit transmission. Most often, these methods adversely affect operations and are implemented for all employees without recognition of different risks posed by different portions of the employee workforce. These broad-based, one-size-fits-all approaches have limited effectiveness and may be cost-prohibitive for operation.

### SUMMARY

In some embodiments a method for workplace disease management is disclosed. The method may include determining employer criteria, screening employees, classifying employees into risk groups, identifying symptomatic employees, establishing work paths and work areas, and monitoring employee compliance with work paths and work areas.

### BRIEF DESCRIPTION OF THE DRAWINGS

The present disclosure is best understood from the following detailed description when read with the accompanying FIGURES. It is emphasized that, in accordance with the standard practice in the industry, various features are not drawn to scale. In fact, the dimensions of the various features may be arbitrarily increased or reduced for clarity of discussion.

The FIGURE is a flowchart of a method in accordance with certain embodiments of the present disclosure.

### DETAILED DESCRIPTION

It is to be understood that the following disclosure provides many different embodiments, or examples, for implementing different features of various embodiments. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

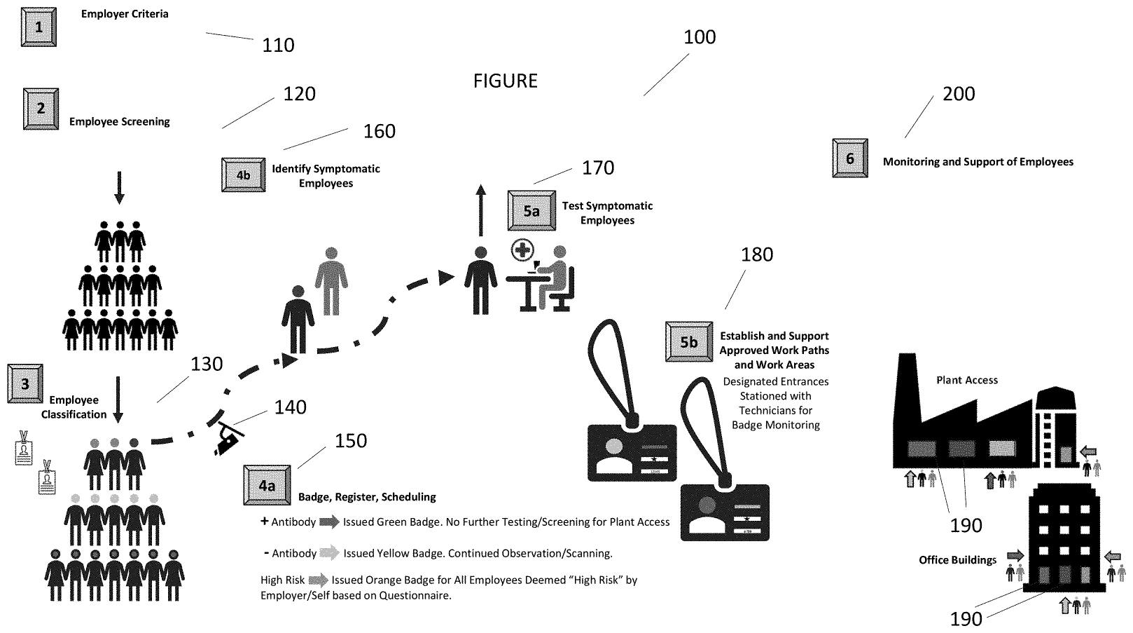

FIG. 1 depicts method for workplace disease management 100 for an employer facility. In certain embodiments, employer criteria 110 are solicited. Such criteria may include, but are not limited to, intended back-to-work date, number of employees at the facility, methods for issuing and screening questionnaires, intentions for high risk employees (such as remaining home, limiting areas, or changing arrival and departure times), contacts and schedules for employee antibody tests, categorization of employees by department, job function, or floor, personal protective equipment desired, position of ingress/egress points and security checkpoints, procedures for working with employer security, existence of parking garages, facility layout, workspaces and controls on those work spaces, floor plans position of common spaces, and desired instructions and protocols such as for guests and employee workflow.

Employees may be screened to determine risk of population groups of employees in employee screening 120. For example, employees may undergo antibody testing, temperature testing 140, or symptom evaluation, and may be issued questionnaires designed to evaluate the risk category of each of the employees. In certain embodiments, all employees entering the facility may be evaluated to determine risk categorization. Examples of questionnaire queries may include employee symptom self-evaluation, recency and detail of travel, previous infection, and exposure to others that are or were infected.

In the embodiment shown in the FIGURE, employees may be classified in employee classification 130 into three risk groups—positive for antibodies, negative for antibodies, and high risk—but the present disclosure contemplates any number of categories and any constitution of the categories. As contemplated by the present disclosure, each risk group may be treated differently in later actions in workplace disease management 100. For example, as shown in the FIGURE, in Badge, Register and Scheduling 150, employees classified as positive for antibodies may be designated, for example, by a badge indicating no further testing or screening is necessary for facility access (shown in the FIGURE as a green badge). Employees classified as negative for antibodies may be designated, for example, by a badge indicating that continued observation and scanning may be required. In addition, employees classified as high risk either by the employer or through answers provided through the questionnaire, may be issued a badge limiting access to the facility, shown in the FIGURE as an orange badge.

Employees that are symptomatic, determined either by a high temperature or through answers to the questionnaire, are identified in identify symptomatic employees 160. Symptomatic employees may be subject to further testing (170) and, based on the results of further testing 170, refused access to the facility, such as being sent home or to a doctor, or deemed non-symptomatic and reclassified.

As part of workplace disease management 100, establish and support approved work paths and work areas 180 may be performed. In this step, entrance stations 190 for different classifications of employees may be established, signage explaining such items as rules and procedures and work paths may be placed, personal protective equipment issued, elevators and stairwells established for different classifications of employees, and restrooms, breakrooms, and conference rooms established for different classifications of employees. In certain embodiments, sanitation tunnels, i.e., tunnels through which UV light or disinfectant may be applied to persons walking through, may be established at entrances stations. Such delineation of different facility areas for different classifications of employees may limit the spread of the infectious agent through the facility.

In monitoring and support of employees (200), employees' badges are verified for entry, departure times, arrival times, and break times are monitored and enforced, and maintenance of hygiene facilities, such as hand sanitizing stations, mask distribution, glove distribution, and sanitizing wipes, is performed. In certain embodiments, all employees are scanned for high temperature at arrival times. In other embodiments, only certain employees, such as those of high risk or negative for antibodies, are scanned for high temperature.

By classifying employees, limiting mingling of different employee classifications, and providing appropriate sanitizing equipment, infections may be limited and efficiency improved.

While a presently preferred embodiment of the invention has been given for purposes of disclosure, numerous changes exist in the details of procedures for accomplishing the desired results. These and other similar modifications will readily suggest themselves to those skilled in the art and are intended to be encompassed within the spirit of the present invention disclosed herein and the scope of the appended claims.

What is claimed is:

1. A method for workplace disease management comprising:
determining employer criteria;
screening employees;
classifying employees into risk groups;
identifying symptomatic employees;
establishing work paths and work areas; and
monitoring employee compliance with work paths and work areas.

2. The method of claim 1, wherein the employer criteria for determine employer criteria is selected from the group consisting of intended back-to-work date, employees at the facility, methods for issuing and screening questionnaires, intentions for high risk employees, contacts and schedules for employee antibody tests, categorization of employees by department, job function, or floor, personal protective equipment desired, position of ingress/egress points and security checkpoints, procedures for working with employer security, existence of parking garages, facility layout, workspaces and controls on the work spaces, floor plans, position of common spaces, desired instructions and protocols such as for guests and employee workflow, and combinations thereof.

3. The method of claim 1, wherein the step of screening employees is performed by antibody testing, temperature testing, symptom evaluation, questionnaire, or a combination thereof.

4. The method of claim 3, wherein the questionnaire includes queries of employee symptom self-evaluation, recency and detail of travel, previous infection, and exposure to others that are or were infected.

5. The method of claim 4, wherein the step of classifying employees is based on information from the step of screen employees.

6. The method of claim 1, wherein employees are classified into three groups.

7. The method of claim 6, wherein the groups comprise positive for antibodies, negative for antibodies, and high risk.

8. The method of claim 1, wherein each risk group is issued a different badge indicating the risk group.

9. The method of claim 1, further comprising when employees are identified as symptomatic, subjecting the employees to further testing.

10. The method of claim 9, wherein based on the results of the further testing, employees are refused access to the facility or deemed non-symptomatic.

11. The method of claim 1, wherein the step of establishing work paths and work areas comprises establishing entrance stations for different classifications of employees, posting signage explaining rules and procedures and work paths, issuing personal protective equipment, designating elevators, stairwells, restrooms, breakrooms, and conference rooms for different classifications of employees, and combinations thereof.

12. The method of claim 1, wherein the step of monitoring employee compliance comprises verifying badges for entry, departure times, arrival times, and combinations thereof.

13. The method of claim 1, wherein the step of monitoring employee compliance comprises maintenance of hygiene facilities.

14. The method of claim 1, wherein sanitation tunnels are established at entrances to the facility.
